(19) United States
(12) Patent Application Publication
(10) Pub. No.: US 2026/0363775 A1
(43) Pub. Date: Nov. 19, 2026

(54) APPARATUS AND METHOD FOR INSERTING PEDICLE SCREW

(71) Applicants: Samuel Kang-Wook Cho, Englewood Cliffs, NJ (US); AJOU UNIVERSITY INDUSTRY-ACADEMIC COOPERATION FOUNDATION, Gyeonggi-do (KR)

(72) Inventors: Sunghyeon Noh, Seoul (KR); Samuel Kang-Wook Cho, Englewood Cliffs, NJ (US)

(21) Appl. No.: 19/210,466

(22) Filed: May 16, 2025

(51) Int. Cl. A61B 34/10; A61B 34/30; G16H 10/60; G16H 40/63

(52) U.S. Cl. CPC A61B 34/10; A61B 34/30; G16H 10/60; G16H 40/63; A61B 2034/107

(57) ABSTRACT

An apparatus and method for inserting a pedicle screw according to an embodiment of the present invention are disclosed. The apparatus may include a memory storing one or more computer-executable instructions and a processor executing the one or more computer-executable instructions, wherein using spinal image data obtained by capturing a spine after inserting the screw into a plurality of patients as training data, the processor is configured to generate an artificial intelligence-based learning model outputting information related to a position of a screw to be inserted into a pedicle in the spine and input spinal image data of a target patient into the trained learning model to output information related to a position of the screw to be inserted into the pedicle of the target patient.

### TECHNICAL FIELD

The present invention relates to an apparatus and method for inserting a pedicle screw, and more particularly, to a pedicle screw-inserting apparatus and method for inserting a screw into a pedicle by learning an artificial neural network learning model.

### RELATED ART

Spinal fusion is a primary treatment for degenerative diseases accompanied by spinal instability. The current standard for spinal fusion is posterior lumbar interbody fusion (PLIF) and pedicle screw fixation. Accurate screw insertion is very important for a successful fusion procedure. Incorrect screw insertion may cause unstable fusion as well as direct damage such as neurovascular injury and bone structure destruction, which may affect the patient's clinical outcome.

The traditional open surgical approach offers the advantage of direct visualization. However, extensive exposure is associated with significant tissue damage including damage to muscles, ligaments and joint structures. This may result in severe postoperative pain and adjacent site degeneration, potentially leading to the need for frequent revision surgeries. Minimally invasive surgery (MIS) technology such as percutaneous pedicle screw fixation is popular to solve these drawbacks.

Ingress points and trajectories for percutaneous screw insertion may be determined based on repeated and continuous fluoroscopic guidance, in conjunction with tactile perception through the puncture needle. These MIS methods utilize an image guidance system, but screw insertion still requires manual operation, which leads to poor screw insertion accuracy. In addition, the increased radiation exposure during continuous fluoroscopic imaging for screw identification raises concerns regarding the health of both the patient and the surgeon.

Robotic-assisted surgery has attracted attention as an alternative treatment method that can overcome the limitations of traditional MIS procedures. The robotic system allows screw insertion accurately without extensive tissue exposure or human intervention, and accurately follows preoperative planning and trajectory planning. This method significantly reduces radiation exposure and minimizes human subjectivity, and as an optimal approach for precise screw insertion, the accuracy of the robotic-assisted surgery has been demonstrated in various studies.

Key technology for surgical robot is software that automatically predicts a direction in which the screw is inserted into the bone and draws an insertion path. Although these studies are currently being actively conducted, in actual clinical settings, a person is still designing the insertion path manually. However, when a person designs the insertion path directly, the screw is often inserted incorrectly.

### SUMMARY

An embodiment of the present invention provides a pedicle screw-inserting apparatus and method for inserting a screw into a pedicle by learning an artificial neural network learning model.

According to an aspect of the present invention, there is provided an apparatus for inserting a pedicle screw including: a memory configured to store one or more computer-executable instructions and a learning model; and a processor configured to execute the one or more computer-executable instructions, wherein the learning model is trained to infer an information related to a position of a screw to be inserted into a pedicle from a spinal image data, and the processor is configured to input a first spinal image data of a target patient into the learning model and output information related to a position of a screw to be inserted into a pedicle of the target patient.

According to an embodiment of the present invention, the learning model may be learned using a second spinal image data in a state in which a screw is inserted into a pedicle, and a training data of the learning model may include a third spinal image data in which an information related to a position of the inserted screw in the second spine image data is marked.

According to an embodiment of the present invention, the training data may further include spinal parameters including at least one of lumbar lordosis (LL), pelvic incidence (PI), pelvic tilt (PT), sacral slope (SS), or sagittal vertical axis (SVA) of the spine included in the third spinal image data.

According to an embodiment of the present invention, the training data may further include a medical historical data including at least one of gender, age, presence or absence of osteoporosis, spinal surgery adaptation, spinal surgery plan, or spinal surgery level.

According to an embodiment of the present invention, the learning model may be a U-Net-based convolutional neural network (CNN) model.

According to an embodiment of the present invention, the information related to the position of the screw may include at least one of coordinate data of the position of the screw, an insertion path of the screw, or an insertion angle of the screw.

According to an embodiment of the present invention, the processor may control a surgical robot to insert the screw into the pedicle of the target patient based on the information related to the position of the screw to be inserted into the pedicle of the target patient.

According to another aspect of the present invention, there is provided a method for inserting a pedicle screw, using a memory configured to store one or more computer-executable instructions and a learning model, and performed by a processor configured to execute the one or more instructions, including: obtaining a first spinal image data of a target patient; inputting the first spinal image data into the learning model; and outputting, using the learning model, information related to a position of a screw to be inserted into a pedicle of the target patient in response to the first spinal image data, wherein the learning model may be trained to infer information related to a position of a screw to be inserted into a spine from a spinal image data.

According to an embodiment of the present invention, the method may further include: securing a second spinal image data in a state in which a screw is inserted into a pedicle; and applying a third spinal image data in which an information related to a position of the inserted screw in the second spine image data is marked as a training data for the learning model.

According to an embodiment of the present invention, the training data may further include spinal parameters including at least one of lumbar lordosis (LL), pelvic incidence (PI), pelvic tilt (PT), sacral slope (SS), or sagittal vertical axis (SVA) of the spine included in the third spinal image data.

According to an embodiment of the present invention, the training data may further include a medical historical data including at least one of gender, age, presence or absence of osteoporosis, spinal surgery adaptation, spinal surgery plan, or spinal surgery level.

According to an embodiment of the present invention, the learning model may be a U-Net-based convolutional neural network (CNN) model.

According to an embodiment of the present invention, the information related to the position of the screw may include at least one of coordinate data of the position of the screw, an insertion path of the screw, or an insertion angle of the screw.

According to an embodiment of the present invention, the method may further include controlling a surgical robot to insert the screw into the pedicle of the target patient based on the information related to the position of the screw to be inserted into the pedicle of the target patient.

According to an embodiment of the present invention, the information related to the position of the third spinal image data may include a first insert position where the screw insertion starts and a second insert position where the inserted screw reaches.

The apparatus and method for inserting a pedicle screw according to an embodiment of the present invention may improve precision and adaptability of a spinal surgical screw insertion.

The apparatus and method for inserting a pedicle screw according to an embodiment of the present invention may improve spinal surgery accuracy, shorten recovery period and hospitalization period of the patient, reduce complications and reoperation, reduce radiation exposure to provide a safe surgical environment, and reduce surgeon's fatigue to shorten overall operative time.

It should be understood that the effects of the present invention are not limited to the above-described effects, and include all effects that can be inferred from the construction of the invention described in the detailed description or the claims.

### BRIEF DESCRIPTION OF THE DRAWINGS

FIG. 1 is a diagram illustrating a pedicle screw-inserting apparatus according to an embodiment of the present invention.

FIG. 2 is a diagram illustrating an artificial neural-based learning model according to an embodiment of the present invention.

FIG. 3 is a diagram illustrating spinal image data according to an embodiment of the present invention.

FIGS. 4 and 5 are experimental data of an example in which a learning model according to an embodiment of the present invention outputs information related to a position where a screw is inserted.

FIGS. 6A and 6B are diagrams illustrating an example of a surgical robot for inserting a screw into a spine of a target patient according to an embodiment of the present invention.

FIG. 7 is a flowchart of a method for inserting a pedicle screw according to an embodiment of the present invention.

### DETAILED DESCRIPTION

Hereinafter, embodiments of the present invention will be described in detail with reference to the accompanying drawings so that those skilled in the art can easily carry out the present invention. The present invention may be implemented in various different forms and is not limited to the embodiments described herein. In order to clearly describe the present invention, parts not related to the description are omitted in the drawings, and the same or similar components are denoted by the same reference numerals throughout the specification.

The words and terms used in the specification and the claims should not be interpreted as limited to ordinary or dictionary meanings, but should be interpreted as meanings and concepts corresponding to technical aspects of the present invention according to principles capable of defining terms and concepts by the inventor in order to best describe the invention.

Therefore, the embodiments described in the specification and the configurations illustrated in the drawings correspond to exemplary embodiments of the present invention and do not represent all technical aspects of the present invention, and therefore, various equivalents and modifications to replace the corresponding configurations may be made at the time of filing the present invention.

In the following description, some components may be omitted to clarify the features of the present invention.

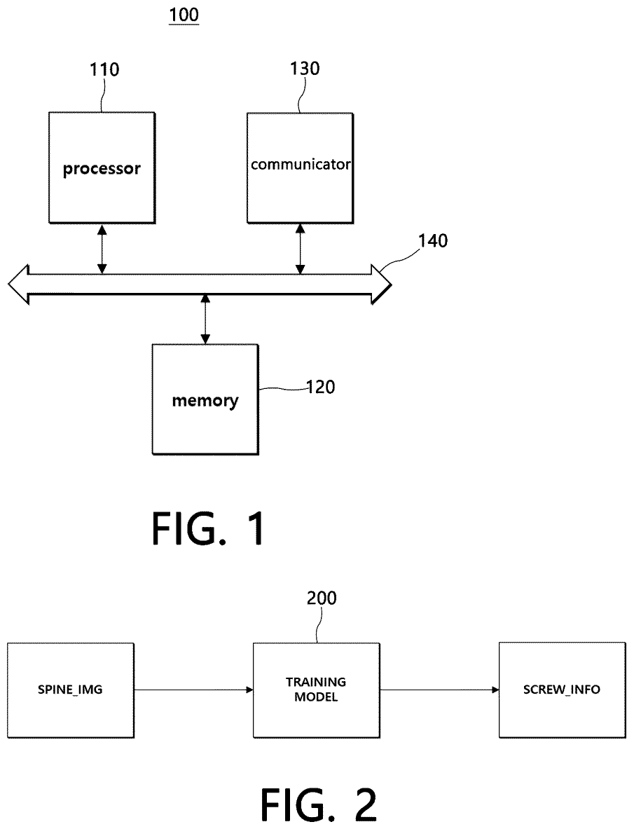

FIG. 1 is a diagram illustrating a pedicle screw-inserting apparatus according to an embodiment of the present invention.

Referring to FIG. 1, the pedicle screw-inserting apparatus 100 may include a processor 110, a memory 120, a transceiver 130, and a communication bus 140. In addition, the pedicle screw-inserting apparatus 100 may further include an input device configured to receive data necessary for generating a learning model for pedicle screw insertion, and an output device configured to output data related to pedicle screw insertion, such as information related to a position at which a screw is inserted, which is output through a learned learning model, although not shown in FIG. 1.

In an embodiment, the processor 110 may execute one or more computer-executable instructions stored in the memory 120.

In an embodiment, the memory 120 may store one or more computer-executable instructions.

In an embodiment, the memory 120 may store data necessary for generating a learning model for pedicle screw insertion.

In an embodiment, the transceiver 130 may transmit and receive data necessary for generating the learning model for pedicle screw insertion by performing information transmission and reception with an external device or a server.

In an embodiment, the processor 110, the memory 120, and the transceiver 130 may be electrically connected to each other through the communication bus 140 to communicate with each other.

In an embodiment, the processor 110 may generate an artificial intelligence-based learning model that outputs information on a position of a screw to be inserted into a pedicle within the spine by using spinal image data that captures the spines of a plurality of patients after screw insertion as training data. For example, the memory 120 may obtain and store the spinal image data of the plurality of patients through the transceiver 130. The processor 110 may generate training data based on the spinal image data, and may input the generated training data to the learning model to train the learning model. In this case, the memory 120 may store instructions for instructing to generate the training data based on the spinal image data, instructions for instructing to generate the artificial intelligence-based learning model, and the like.

In an embodiment, the spinal image data may be image data generated by imaging using techniques such as computed tomography (CT), X-ray, magnetic resonance imaging (MRI).

In an embodiment, the training data may further include medical history data including at least one of gender, age, presence or absence of osteoporosis, spinal surgery adaptation, spinal surgery plan, and spinal surgery level. In this case, the processor 110 may learn the learning model using the spinal image data and the medical history data as the training data. In addition, the memory 120 may store training data such as spinal image data, medical history data, or the like.

In an embodiment, the processor 110 may input the spinal image data of the target patient to the trained learning model and output information related to the position of the screw to be inserted into the pedicle of the target patient. For example, the memory 120 may obtain and store the spinal image data of the target patient through the transceiver 130. The processor 110 may output information related to the position of the screw to be inserted into the pedicle of the target patient by inputting the spinal image data of the target patient into the trained learning model.

In an embodiment, the processor 110 may control the surgical robot to insert the screw into the pedicle of the target patient based on the information related to the position of the screw to be inserted into the pedicle of the target patient. For example, the processor 110 may provide the surgical robot with the information related to the position of the screw to be inserted into the pedicle of the target patient and a control signal instructing the surgical robot to insert the screw.

In an embodiment, the surgical robot may be implemented as a configuration of the pedicle screw-inserting apparatus 100 although not illustrated in FIG. 1. As another example, the surgical robot may be an external device that communicates with the pedicle screw-inserting apparatus 100 through the transceiver 130.

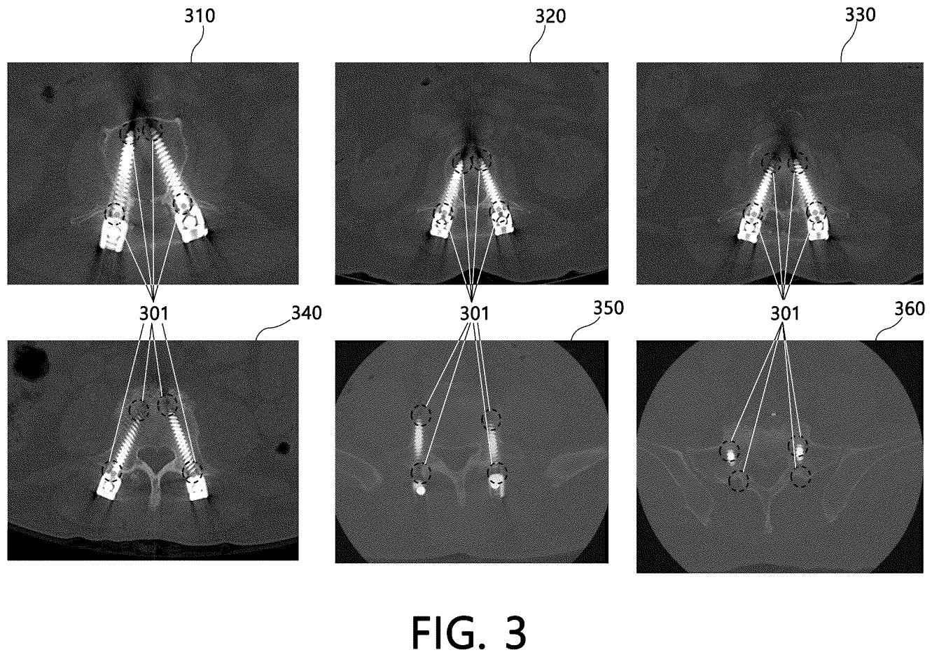

FIG. 2 is a diagram illustrating an artificial neural-based learning model according to an embodiment of the present invention.

Referring to FIG. 2, the artificial neural-based learning model 200 may be a U-Net-based convolutional neural network (CNN) model. However, this is merely an example, and the learning model 200 may be implemented as a neural network model of various structures capable of processing images.

In an embodiment, the learning model 200 may be learned using spinal image data (SPINE_IMG) as training data. In this case, the training data may further include medical history data. In this case, the learning model 200 may be trained using both the spinal image data (SPINE_IMG) and the medical history data as training data.

The spinal image data (SPINE_IMG) may be any one of an X-ray image, an MRI image, and a CT image, but is not necessarily limited thereto. The spinal image data (SPINE_IMG) includes information capable of displaying a shape of the spine as two-dimensional or three-dimensional information.

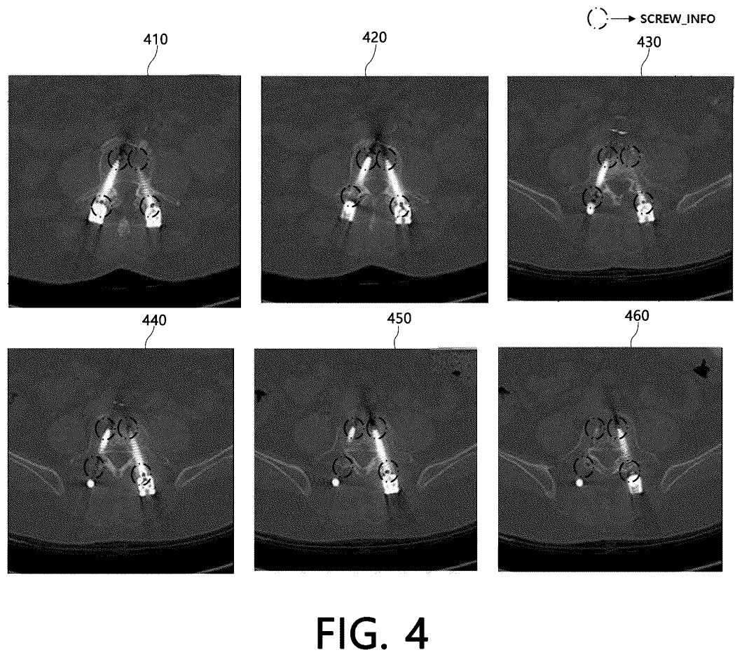

The spinal image data (SPINE_IMG) may also be an image including screw information. At this time, the spinal image data (SPINE_IMG) may also include information related to spinal parameters. In this case, the learning model 200 is trained to infer a corresponding position after learning a relationship between the spinal image and a labeled position. The spinal image data (SPINE_IMG) for training may be labeled with screw position information by an operator. For example, the position within the circular dotted line in FIG. 3 may be marked with a red circle.

In an alternative embodiment, the spinal image data (SPINE_IMG) may not include information related to the screw. The screw information that is not included may be information related to the screw to be inserted later, and it is also possible to include information related to the screw that has already been inserted. The information related to the screw to be inserted corresponds to information to be inferred from the input data. In this case, a correlation between the spinal image before the screw is inserted and the spinal image after the screw is inserted may be trained.

In an embodiment, the learning model 200 may be trained to receive the spinal image data (SPINE_IMG) as input and output information (SCREW_INFO) related to the position of the screw to be inserted into the pedicle. To this end, the training data may include the spinal image data (SPINE_IMG), medical history data, along with screw position information data for the same individual. The screw position information data may either be direct information related to the screw position itself or a spinal image in which a screw have already been inserted, allowing the screw position to be inferred through machine learning.

In an embodiment, the learning model 200 may be trained to infer information (SCREW_INFO) related to the position of the screw to be inserted into the pedicle based on the spinal image data (SPINE_IMG).

In an embodiment, the learning model 200 may be trained to infer information (SCREW_INFO) related to the position of the screw to be inserted into the pedicle based on the spinal image data (SPINE_IMG) and the medical history data.

In an embodiment, the information (SCREW_INFO) related to the position of the screw may include at least one of coordinate data of the position of the screw, an insertion path of the screw, or an insertion angle of the screw. The coordinate data may represent coordinates related to the position where the screw is inserted in the spinal image data. The insertion path may represent a path through which the screw is inserted in the spinal image data. The insertion angle may represent an angle through which the screw is inserted in the spinal image data.

The coordinate data of the screw position may include at least some information specifying a relative positional relationship with the spine when the screw is inserted into the spine, such as one side and the other side of the screw or the center point and the angle.

The insertion path of the screw may be specified as a first insert position where the screw insertion starts and a second insert position where the inserted screw reaches (indicated by the circular dotted line in FIG. 3).

The insertion angle of the screw may mean an angle formed between a predetermined line or plane and the screw body when the screw is inserted. The insertion angle of the screw may supplement information that is difficult to recognize as a 2D image.

In an embodiment, the trained learning model 200 may receive the spinal image data (SPINE_IMG) of the target patient as input and may output information (SCREW_INFO) related to the position of the screw to be inserted into the pedicle of the target patient.

In an embodiment, the trained learning model 200 may additionally receive the medical history data of the target patient in addition to the spinal image data (SPINE_IMG) of the target patient as input and may output information (SCREW_INFO) related to the position of the screw to be inserted into the pedicle of the target patient. In this case, the accuracy of the information (SCREW_INFO) related to the position of the screw may be increased.

Accordingly, the pedicle screw-inserting apparatus 100 according to an embodiment of the present disclosure may accurately and automatically generate the insertion path of the screw through the learning model 200.

FIG. 3 is a diagram illustrating spinal image data according to an embodiment of the present invention.

Referring to FIG. 3, the first spinal image data 310 may be image data generated by capturing a first lumbar vertebra, which is part of a spine. The first lumbar vertebra may be medically referred to as L1. The second spinal image data 320 may be image data generated by capturing a second lumbar vertebra, which is part of a spine. The second lumbar vertebra may be medically referred to as L2. The third spinal image data 330 may be image data generated by capturing a third lumbar vertebra, which is part of a spine. The third lumbar vertebra may be medically referred to as L3. The fourth spinal image data 340 may be image data generated by capturing a fourth lumbar vertebra, which is part of a spine. The fourth lumbar vertebra may be medically referred to as L4. The fifth spinal image data 350 may be image data generated by capturing a fifth lumbar vertebra, which is part of a spine. The fifth lumbar vertebra may be medically referred to as L5. The sixth spinal image data 360 may be image data generated by capturing a sixth lumbar vertebra, which is part of a spine. The sixth lumbar vertebra may be medically referred to as L1.

In an embodiment, the plurality of spinal image data 310 to 360 may be preprocessed before being input to the learning model 200.

In an embodiment, the plurality of spinal image data 310 to 360 may be preprocessed to include information 301 related to a position where a screw is inserted. For example, the plurality of spinal image data 310 to 360 may be image data generated by capturing the screw after being inserted into the pedicle. In this case, the plurality of spinal image data 310 to 360 may be preprocessed so that the information 301 related to the positions where screws have been inserted are labeled. Specifically, the center coordinates, the insertion path, and the insertion angle of the screw may be labeled in the plurality of spinal image data 310 to 360. Accordingly, the plurality of spinal image data 310 to 360 may include the information 301 related to the positions where the screws are inserted.

In an embodiment, the plurality of spinal image data 310 to 360 may be preprocessed to include spinal parameters including at least one of lumbar lordosis (LL), pelvic incidence (PI), pelvic tilt (PT), sacral slope (SS), and sagittal vertical axis (SVA) related to the spine. Accordingly, the plurality of spinal image data 310 to 360 may include information related to the spinal parameters. The spinal parameters may refer to various measurement values used to evaluate alignment state and balance of the spine. The parameters may play an important role in evaluating a health state of the spine and diagnosing a spine deformation or disease. For example, lumbar lordosis may be an angle measuring a lordotic curve of the lumbar vertebra, pelvic incidence angle may be an angle between a line connecting a top of the sacrum and the center of a hip joint and a vertical line of the top of the sacrum, pelvic tilt may be an angle between a line connecting the center of the hip joint and the top of the sacrum and the vertical line, sacral inclination angle may be an angle between the top of the sacrum and a horizontal line, and the sagittal vertical axis may be the vertical distance between the seventh cervical vertebra and the top of the sacrum. These parameters are used to evaluate the sagittal alignment and balance of the spine, and may provide important information for diagnosing spinal deformities or conditions and for establishing treatment plans.

In an embodiment, the preprocessing of the plurality of spinal image data 310 to 360 may be performed through a user input, control, or the like. As another example, the preprocessing of the plurality of spinal image data 310 to 360 may be performed by the processor 110 through various techniques determined in advance.

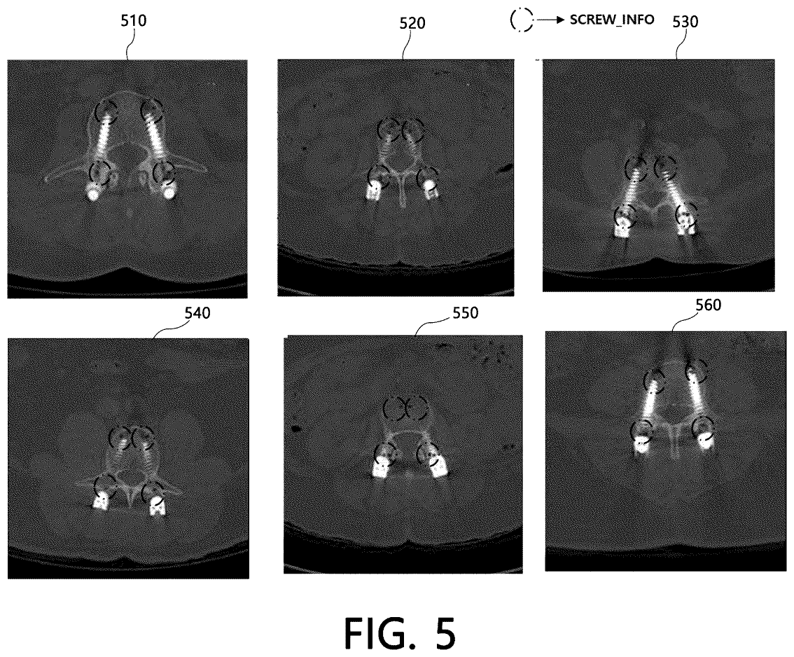

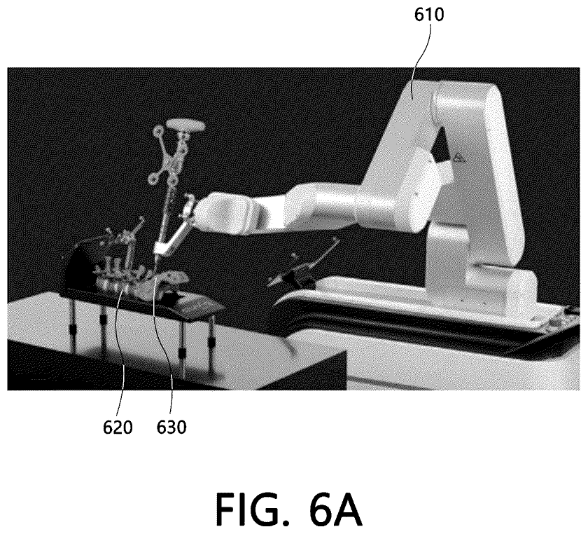

FIGS. 4 and 5 are experimental data of an example in which a learning model according to an embodiment of the present invention outputs information related to a position where a screw is inserted.

Referring to FIGS. 4 and 5, the processor 110 may input a plurality of spinal image data 410 to 460 and 510 to 560 of a target patient into the learned learning model 200. The learning model 200 may analyze a plurality of spinal image data 410 to 460 and 510 to 560 of the target patient and output information (SCREW_INFO) related to a position of a screw to be inserted into a pedicle.

For example, the processor 110 may output information (SCREW_INFO) related to the position of the screw to be inserted into the pedicle within each of the plurality of spinal image data 410 to 460 and 510 to 560. In this case, the information (SCREW_INFO) related to the position of the screw may include at least one of coordinate data of the position of the screw, an insertion path of the screw, and an insertion angle of the screw.

As shown in FIGS. 4 and 5, the information (SCREW_INFO) related to the position of the screw output through the learned learning model 200 may have a very high accuracy compared to the position of the screw inserted into the actual pedicle.

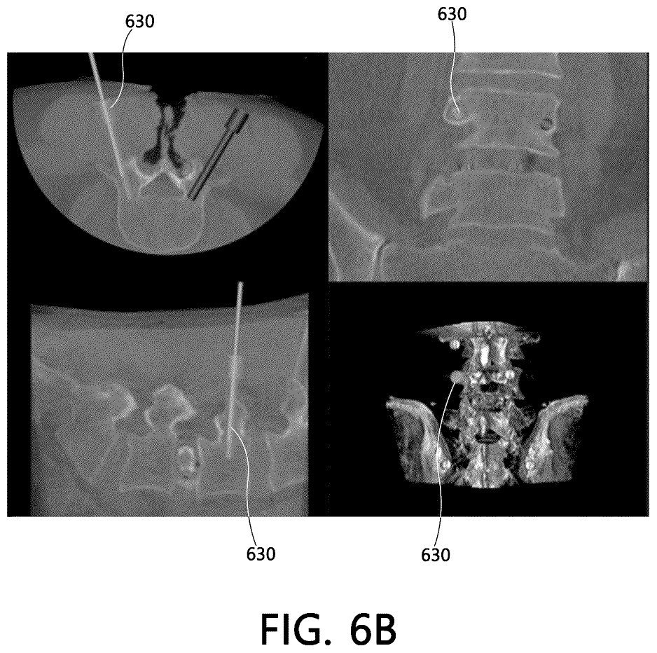

FIGS. 6A and 6B are diagrams illustrating an example of a surgical robot for inserting a screw into a spine of a target patient according to an embodiment of the present invention.

Specifically, FIG. 6A is a diagram illustrating the appearance of a surgical robot for inserting a screw into a spine, and FIG. 6B is a diagram illustrating an image of a state in which a screw is inserted into a spine. In FIG. 6B, the image shown at the top left is a view of the spine taken from above, the image shown at the bottom left is a view of the spine taken from the side, and the diagrams shown on the right side may view of the spine taken from the front or back.

Referring to FIGS. 6A and 6B, the surgical robot 610 may include a robot arm for inserting a pedicle screw.

In an embodiment, the surgical robot 610 may be implemented as a configuration of the pedicle screw-inserting apparatus 100 although not illustrated in FIG. 1. As another example, the surgical robot 610 may be an external device that communicates with the pedicle screw-inserting apparatus 100 through the transceiver 130.

In an embodiment, the surgical robot 610 may insert the screw 630 into the spine 620 of the target patient based on the information related to the position of the screw output through the trained learning model 200. For example, the surgical robot 610 may insert the screw 630 into the spine 620 of the target patient by referring to the coordinate data, the insertion path, the insertion angle, and the like included in the information related to the position of the screw 630 output through the learning model 200.

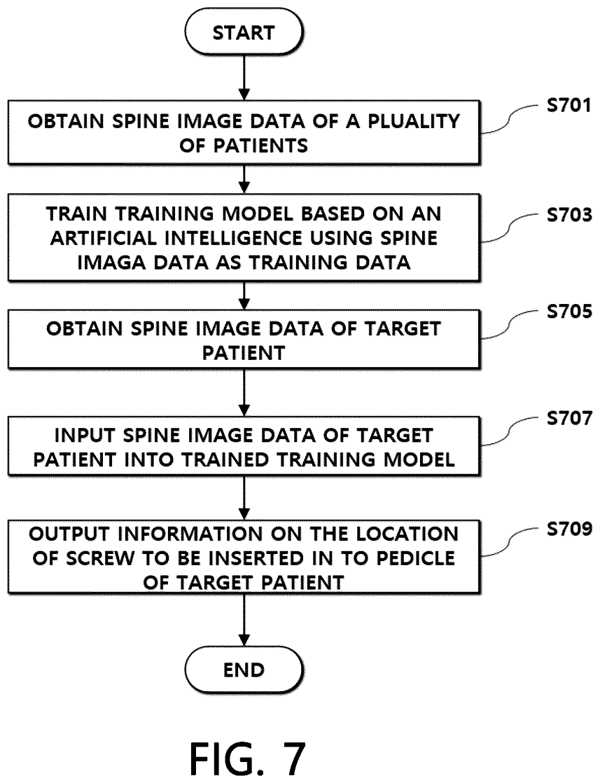

FIG. 7 is a flowchart of a method for inserting a pedicle screw according to an embodiment of the present invention.

The method illustrated in FIG. 7 may be performed by, for example, the pedicle screw-inserting apparatus 100 illustrated in FIG. 1.

In step S701, the pedicle screw-inserting apparatus 100 may obtain spinal image data that captures the spines of a plurality of patients after screw insertion.

In step S703, the pedicle screw-inserting apparatus 100 may trained an artificial intelligence-based learning model that outputs information related to a position of a screw to be inserted into a pedicle within the spine, using the spinal image data as training data.

In step S705, the pedicle screw-inserting apparatus 100 may obtain spinal image data of a target patient.

In step S707, the pedicle screw-inserting apparatus 100 may input the spinal image data of the target patient into the trained learning model.

In step S709, the pedicle screw-inserting apparatus 100 may output information related to the position of the screw to be inserted into the pedicle of the target patient, using the trained learning model into which the spinal image data of the target patient is input.

In addition, the pedicle screw-inserting apparatus 100 may control the surgical robot to insert the screw into the pedicle of the target patient based on the information related to the position of the screw to be inserted into the pedicle of the target patient.

Meanwhile, according to an embodiment, each step illustrated in FIG. 7 may be changed, added, or removed in order.

Although the embodiments of the present invention are described, the spirit of the present invention is not limited by the embodiments presented in the specification, and those skilled in the art who understand the spirit of the present invention may easily propose other embodiments by adding, modifying, deleting, and adding components within the same spirit, but these are also within the spirit of the present invention.

What is claimed is:

1. An apparatus for inserting a pedicle screw comprising:
a memory configured to store one or more computer-executable instructions and a learning model; and
a processor configured to execute the one or more computer-executable instructions,
wherein the learning model is trained to infer an information related to a position of a screw to be inserted into a pedicle from a spinal image data, and
the processor is configured to input a first spinal image data of a target patient into the learning model and output information related to a position of a screw to be inserted into a pedicle of the target patient.

2. The apparatus of claim 1, wherein the learning model is learned using a second spinal image data in a state in which a screw is inserted into a pedicle, and
a training data of the learning model includes a third spinal image data in which an information related to a position of the inserted screw in the second spine image data is marked.

3. The apparatus of claim 2, wherein the training data further comprises spinal parameters including at least one of lumbar lordosis (LL), pelvic incidence (PI), pelvic tilt (PT), sacral slope (SS), or sagittal vertical axis (SVA) of the spine included in the third spinal image data.

4. The apparatus of claim 2, wherein the training data further comprises a medical historical data including at least one of gender, age, presence or absence of osteoporosis, spinal surgery adaptation, spinal surgery plan, or spinal surgery level.

5. The apparatus of claim 1, wherein the learning model is a U-Net-based convolutional neural network (CNN) model.

6. The apparatus of claim 1, wherein the information related to the position of the screw comprises at least one of coordinate data of the position of the screw, an insertion path of the screw, or an insertion angle of the screw.

7. The apparatus of claim 1, wherein the processor is configured to control a surgical robot to insert the screw into the pedicle of the target patient based on the information related to the position of the screw to be inserted into the pedicle of the target patient.

8. A method for inserting a pedicle screw, using a memory configured to store one or more computer-executable instructions and a learning model, and performed by a processor configured to execute the one or more instructions, comprising:
obtaining a first spinal image data of a target patient;
inputting the first spinal image data into the learning model; and
outputting, using the learning model, information related to a position of a screw to be inserted into a pedicle of the target patient in response to the first spinal image data,
wherein the learning model is trained to infer information related to a position of a screw to be inserted into a spine from a spinal image data.

9. The method of claim 8, further comprising:
securing a second spinal image data in a state in which a screw is inserted into a pedicle; and
applying a third spinal image data in which an information related to a position of the inserted screw in the second spine image data is marked as a training data for the learning model.

10. The method of claim 9, wherein the training data further comprises spinal parameters including at least one of lumbar lordosis (LL), pelvic incidence (PI), pelvic tilt (PT), sacral slope (SS), or sagittal vertical axis (SVA) of the spine included in the third spinal image data.

11. The method of claim 9, wherein the training data further comprises a medical historical data including at least one of gender, age, presence or absence of osteoporosis, spinal surgery adaptation, spinal surgery plan, or spinal surgery level.

12. The method of claim 8, wherein the learning model is a U-Net-based convolutional neural network (CNN) model.

13. The method of claim 8, wherein the information related to the position of the screw comprises at least one of coordinate data of the position of the screw, an insertion path of the screw, or an insertion angle of the screw.

14. The method of claim 8, further comprising:
controlling a surgical robot to insert the screw into the pedicle of the target patient based on the information related to the position of the screw to be inserted into the pedicle of the target patient.

15. The method of claim 9, wherein the information related to the position of the third spinal image data comprises a first insert position where the screw insertion starts and a second insert position where the inserted screw reaches.
